(19) United States
(12) Patent Application Publication
(10) Pub. No.: US 2026/0076873 A1
(43) Pub. Date: Mar. 19, 2026

(54) Closed-System Infusion Adapter with Simplified Connection Mechanism

(71) Applicant: Becton, Dickinson and Company, Franklin Lakes, NJ (US)

(72) Inventors: Linda Kunardi, Singapore (SG); Ramon Alaiz Chueca, Madrid (ES)

(21) Appl. No.: 18/885,880

(22) Filed: Sep. 16, 2024

(51) Int. Cl. A61J 1/20; A61J 1/10; A61J 1/14; A61M 5/162

(52) U.S. Cl. CPC A61J 1/201; A61J 1/1443; A61J 1/2048; A61J 1/2096; A61J 1/10; A61M 5/162

(57) ABSTRACT

An infusion adapter for connection with an infusion fluid container is provided. The infusion adapter includes a connection spike positioned at a first end of the infusion adapter, a connection stick positioned at a second end of the infusion adapter and arranged orthogonal to the connection spike, and a joining member connecting the connection spike to the connection stick. The connection spike has a main spike portion and a puncturing point configured to engage an injection port of an infusion fluid container. The connection stick includes a locking mechanism configured to connect to an external component via a push-type engagement, with the locking mechanism including a biasing member and an engagement member. A fluid path extends through the joining member, the connection spike, and the connection stick, to provide for a transfer of fluid from the second end to the first end.

### BACKGROUND OF THE INVENTION

### Field of the Invention

The present disclosure relates generally to an infusion adapter and, more particularly, to a closed-system infusion adapter configured to attach with an external component in a simplified manner for a drug transfer procedure.

### Description of Related Art

Intravenous therapy applications allow patients to receive infusion and medication treatment. For example, therapy may include the administration of medications by IV using intravenous and subcutaneous or hypodermis routes, i.e., into the bloodstream and under the skin. Examples of medical treatments that intravenous therapy applications may provide to a patient include antibiotics, pain management medications, cancer treatments, and similar medications.

Medications may be packaged as “pre-filled” devices, wherein a syringe assembly is pre-filled with medication prior to being packaged and delivered to a patient. “Pre-filled” devices eliminate the need for a user to fill the device prior to injection.

Certain drugs or medications are preferably provided in powder or dry form (such as a lyophilized form) and require reconstitution prior to administration. Lyophilized drugs, for example, typically are supplied in a freeze-dried form that needs to be mixed with a diluent to reconstitute the substance into a form that is suitable for injection. In addition, drugs may be provided as multipart systems that require mixing prior to administration. For example, one or more liquid components, such as flowable slurries, and one or more dry components, such as powdered or granular components, may be provided in separate containers that require mixing prior to administration.

A patient may be provided with an intravenous system that includes intravenous tubing and a connector that is adapted to receive an injector and/or syringe assembly containing a required medication. In this manner, when a treatment is needed, a patient or a medical practitioner is able to connect a syringe assembly to the connector and then inject a medication intravenously into the patient via the injector and/or syringe assembly, the connector, and the intravenous tubing.

When performing infusion, it is often necessary to inject a drug or other medical substance into the infusion fluid inside an infusion bag or other infusion fluid container. This is often done by means of penetrating a septum or other fluid barrier of an injection port on the infusion bag, and this may be done by an infusion adapter having a spike connector thereon, with a syringe in-turn connected to the infusion adapter that is filled with the medical fluid in question.

It is recognized that existing infusion adapters may be cumbersome for healthcare providers to use and/or suffer from performance issues when used. As one example, the interfacing of an external component (e.g., syringe) to such an infusion adapter may require a multi-step connection process-including engaging the component with the infusion adapter, rotating the component relative to the infusion adapter, and finally pushing the component further into the infusion adapter. As another example, for L-shaped infusion adapters, the adapter must have sufficient structural integrity to be able to resist bending as the adapter is inserted/spiked into an IV bag through the rubber bung thereof.

Accordingly, it is desirable to provide an infusion adapter that may be attached to an external component, such as a syringe, in a simplified manner. It is also desirable for such an infusion adapter to have sufficient rigidity to resist bending or other deformation during use thereof, with the infusion adapter having such rigidity without the size or bulk of the adapter being increased.

### SUMMARY OF THE INVENTION

Provided herein is an infusion adapter for connection with an infusion fluid container. The infusion adapter includes a connection spike positioned at a first end of the infusion adapter, with the connection spike having a main spike portion and a puncturing point configured to engage an injection port of an infusion fluid container. The infusion adapter also includes a connection stick positioned at a second end of the infusion adapter and arranged orthogonal to the connection spike, the connection stick comprising a locking mechanism including a biasing member and an engagement member, with the locking mechanism configured to connect to an external component via a push-type engagement. The infusion adapter further includes a joining member connecting the connection spike to the connection stick in the orthogonal arrangement, the joining member integrally formed with the connection spike and the connection stick. A fluid path is formed through the joining member, the connection spike, and the connection stick, extending from the first end to the second end, to provide for a transfer of fluid injected from the second end to the first end.

In some embodiments, the biasing member comprises a pair of cantilever arms and the engagement member comprises a projection extending radially outwards from each of the cantilever arms, the pair of cantilever arms configured to deflect radially inward during engagement of the connection stick to the external component.

In some embodiments, the joining member is structured as an L-shaped member and comprises a first segment aligned with and joined to the connection spike and a second segment aligned with and joined to the connection stick, wherein the first segment and second segment are connected at a junction.

In some embodiments, the connection spike comprises an end flange on an end thereof opposite the connection spike, and wherein the first segment comprises a central column joined to the end flange, with the central column including a plurality of reinforcement ribs formed thereon that extend radially outward from the central column, the reinforcement ribs increasing a mechanical strength a connection between the joining member and the connection spike.

In some embodiments, the plurality of reinforcement ribs comprises a first rib and a second rib positioned on opposing sides of the central column, with each of the first rib and the second rib having a wedge-shaped construction.

In some embodiments, the first segment comprises a rest plate located along a length of the first segment between the plurality of reinforcement ribs and the junction, the rest plate oriented in parallel with the end flange and spaced apart therefrom by a gap provided by the central column and the plurality of reinforcement ribs.

In some embodiments, the infusion adapter comprises a spike cap positioned over the connection spike, to protect the puncturing point, and wherein the rest plate provides a datum for the spike cap, to ensure proper connection of the spike cap onto the connection spike.

In some embodiments, the gap is sized to allow for entry of a sterilizing gas into an interior of the spike cap with the spike cap positioned over the connection spike, so as to provide for sterilizing of the connection spike.

In some embodiments, the second segment comprises an elongated portion and a flanged divider, the elongated portion generally extending a full length of the second segment, with one end thereof joined with the first segment and another end thereof joined with the flanged divider.

In some embodiments, the flanged divider is affixed to the elongated portion on one face thereof and to the connection stick on an opposing face thereof.

In some embodiments, the elongated portion comprises a tapered member that flares outward along a length thereof as the elongated portion extends from the end thereof joined with the first segment to the end thereof joined with the divider flange.

In some embodiments, a thickness of an outer wall of the elongated portion increases along the length thereof as the elongated portion extends from the end thereof joined with the first segment to the end thereof joined with the divider flange.

In some embodiments, the connection stick comprises a membrane seat that retains a membrane therein, the membrane providing for a closed transfer of fluid when the connection stick is connected to the external component.

In some embodiments, the infusion adapter comprises an integrally formed, molded adapter.

Also provided herein is a fluid transfer system that includes an infusion fluid container containing an infusion fluid therein, the infusion fluid container comprising a first port and a second port each having a fluid barrier member therein, an intravenous tubing connected to the second port such that the intravenous tubing is in fluid communication with the infusion fluid container, an infusion adapter having a connection spike engaged with the first port such that the infusion adapter is in fluid communication with the infusion fluid container, and a syringe assembly coupled to the connection stick, the syringe assembly comprising a pre-filled syringe containing medication fluid and a syringe adapter, with the connection stick engaged with the syringe adapter.

In some embodiments, the fluid barrier member comprises a rubber bung.

In some embodiments, the syringe adapter comprises a housing defining a cavity within which the locking mechanism is received upon connection of the connection stick with the syringe adapter.

In some embodiments, the syringe adapter comprises another membrane, the another membrane seated in the cavity of the housing and configured to engage the membrane of the connection stick upon connection of the syringe assembly to the infusion adapter, with the engaging of the membrane and the another membrane providing a substantially leak-free connection of the infusion adapter with the syringe adapter during transfer of the medication fluid.

### BRIEF DESCRIPTION OF THE DRAWINGS

FIG. 1 is perspective view of an infusion adapter, according to one aspect of the present disclosure;

FIG. 2 is a front view of the infusion adapter of FIG. 1;

FIG. 3 is a cross-sectional view of the infusion adapter of FIG. 2;

FIG. 4 is a perspective view of area A of the infusion adapter of FIG. 1;

FIG. 5 is a cross-sectional view taken along line 5-5 of FIG. 4;

FIG. 6 is a perspective view of area A of the infusion adapter of FIG. 1;

FIG. 7 is a cross-sectional view taken along line 7-7 of FIG. 6; and

FIG. 8 is a perspective view of a fluid transfer system, according to one aspect of the present disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

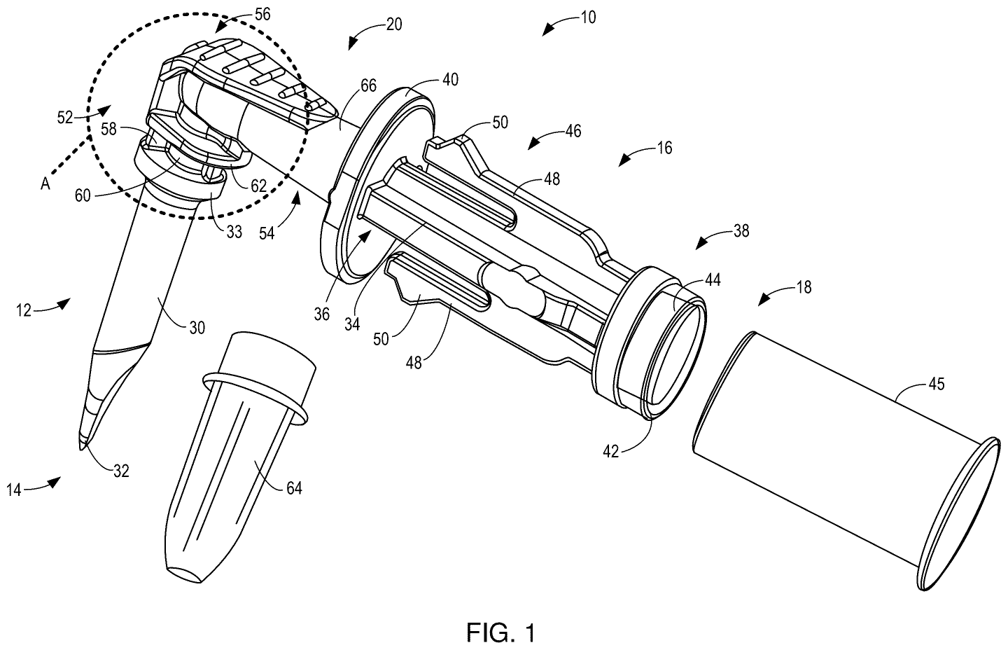

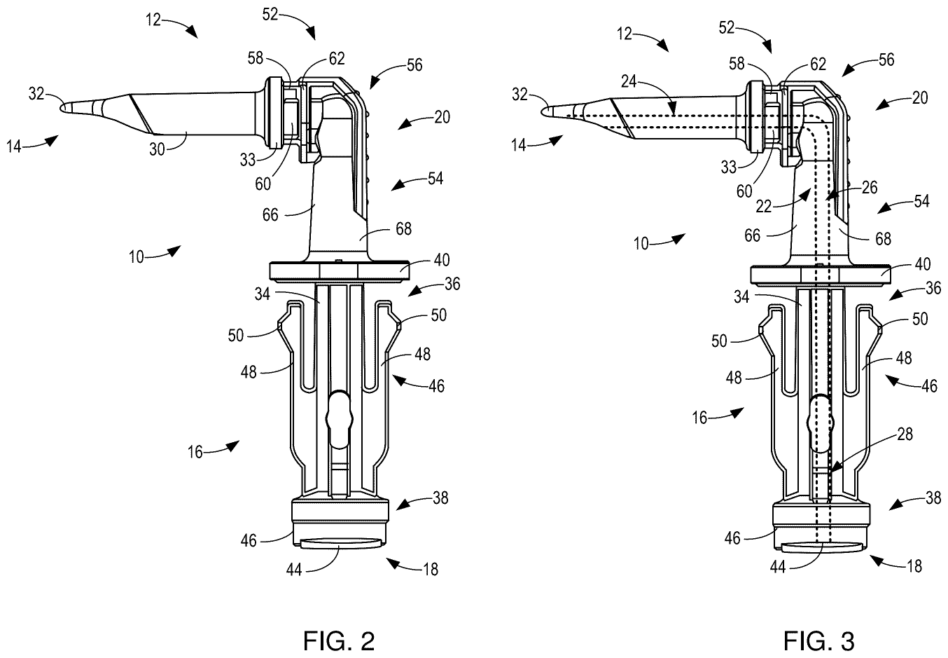

Referring to FIGS. 1-7, shown is a non-limiting embodiment of an infusion adapter 10 used in/with the closed preparation of a drug. The infusion adapter 10 includes a connection spike 12 located at a first end 14 of the adapter 10 and a connection stick 16 located at a second end 18 of the adapter 10. The infusion adapter 10 also includes a joining member 20 connecting the connection spike 12 with the connection stick 16, with the joining member 20 orienting the connection spike 12 and connection stick 16 in an orthogonal arrangement such that the infusion adapter 10 may be generally configured as a “L-shape” adapter or connector. The infusion adapter 10 may be provided as a single, integrally-formed component, with the infusion adapter 10 formed via an injection molding process, as a non-limiting example. In some embodiments, the infusion adapter 10 may thus be formed of a thermoplastic or thermosetting polymer, as non-limiting examples.

As shown best in FIG. 3, a fluid path 22 is formed through the infusion adapter 10, with a first fluid channel 24 formed in connection spike 12 that is fluidly connected to a second fluid channel 26 formed in joining member 20 and a third fluid channel 28 formed in connection stick 16. The infusion adapter 10 may be connected to a syringe assembly containing a medication fluid via the connection stick 16 and may be connected to an intravenous fluid bag via connection spike 12, with the fluid path 22 providing for a transfer of medication fluid from the syringe assembly to the intravenous fluid bag—i.e., a medication fluid may flow into the infusion adapter 10 via the connection stick 16 at second end 18 and travel through the fluid path 22 and out the spike connection 12 at first end 14.

As shown in FIGS. 1-3, connection spike 12 includes an elongated cylindrical main spike portion 30, a puncturing point 32 disposed adjacent first end 14, and an end flange 33 opposite from puncturing point 32. In some embodiments, an anchor component (not shown) may be provided on main spike portion 30, such as an annular retaining ring or helical thread, that provides additional retention of the infusion adapter 10 with the port of an intravenous fluid bag. The first fluid channel 24 extends through a length of the main spike portion 30—extending from the puncturing point 32 at first end 14 to a junction/connection with second fluid channel 26 in joining member 20.

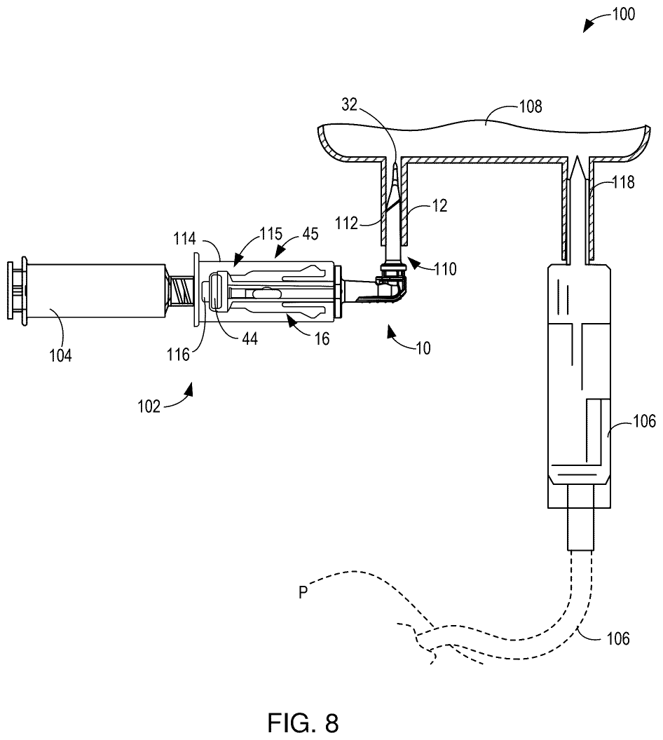

Connection stick 16 includes a body 34 having a first end 36 and a second end 38, with the body 34 defining the third fluid channel 28 that extends therethrough. The first end 36 of body 34 may be affixed with the joining member 20, with the body 34 extending out from a flanged divider 40 formed on joining member 20. The second end 38 of the connection stick 16 may include a membrane seat 42 that is defined by a portion of the body 34 of the connection stick 16, with the membrane seat 42 recessed relative to the second end 38 of the body 34 of the connection stick 16. The membrane seat 42 is configured to receive a membrane 44 therein. The membrane 44 of the connection stick 16 may be configured to engage a membrane of a syringe adapter 45 used to attach the infusion adapter 10 to a pre-filled syringe (FIG. 8), with the engaging of the membrane 44 and the syringe adapter 45 membrane providing a substantially leak-free connection with the syringe adapter 45 during fluid transfer.

As shown in FIGS. 1-3, body 34 of the connection stick 16 includes a locking mechanism 46 having a biasing member 48 and an engagement member 50 positioned on the biasing member 48—with the locking mechanism 46 allowing for a simple push-type connection of the infusion adapter 10 with the syringe adapter 45. According to embodiments, the biasing member 48 of the locking mechanism 46 comprises a pair of cantilever arms and the engagement member 50 comprises a projection extending radially outwards from each of the cantilever arms. The engagement members 50 are configured to engage a portion of a housing of the syringe adapter 45 to bias the engagement member 50 radially inward via the cantilever arms during insertion of the connection stick 16 into the syringe adapter 45. The engagement members 50 are also configured to return to a non-biased position when the connection stick 16 has been fully inserted into the syringe adapter 45 to secure the infusion adapter 10 to the syringe adapter 45. Accordingly, upon inserting connection stick 16 into the housing of the syringe adapter 45, the biasing members 48 are deflected radially inward through engagement of the engagement members 50 with the housing. Once fully inserted, the biasing members 48 return to the non-biased position with the engagement members 50 retaining the connection stick 16 in the syringe adapter 45. To remove the infusion adapter 10, a user applies an axial force to the infusion adapter 10 in a direction away from the syringe adapter 45 such that the biasing members 48 are again deflected radially inward through engagement of the engagement members 50 with the housing of the syringe adapter 45 until the engagement members 50 are axially displaced beyond the housing of the syringe adapter 45.

As shown in FIGS. 1-3, the joining member 20 is structured as an L-shaped member having a first segment 52 and a second segment 54 that are oriented orthogonally to one another. The first segment 52 is joined with the connection spike 12 and is aligned therewith, while the second segment 54 is joined with the connection stick 16 and is aligned therewith. The first segment 52 may be joined to second segment 54 at a junction 56. According to aspects of the disclosure, the joining member 20 mechanically and fluidly connects the connection spike 12 and connection stick 16 of the infusion adapter 10, while also being designed to provide mechanical strength to the infusion adapter 10 and provide for simplified manufacturing of the infusion adapter 10.

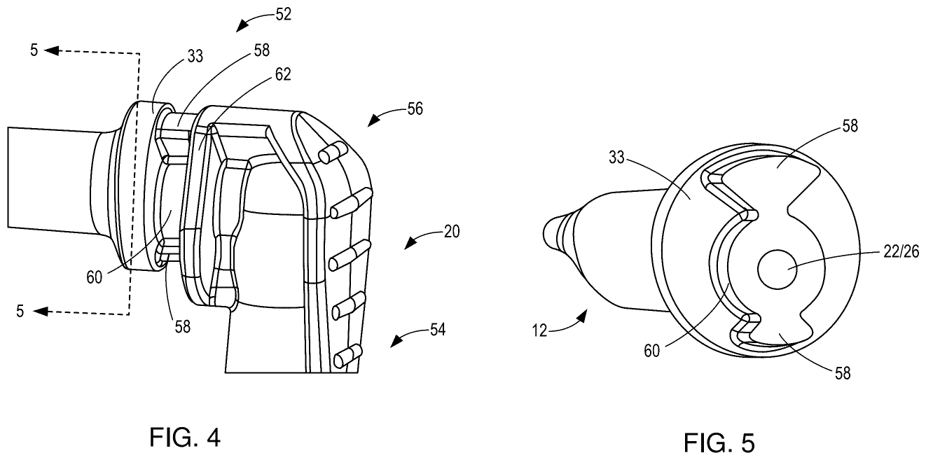

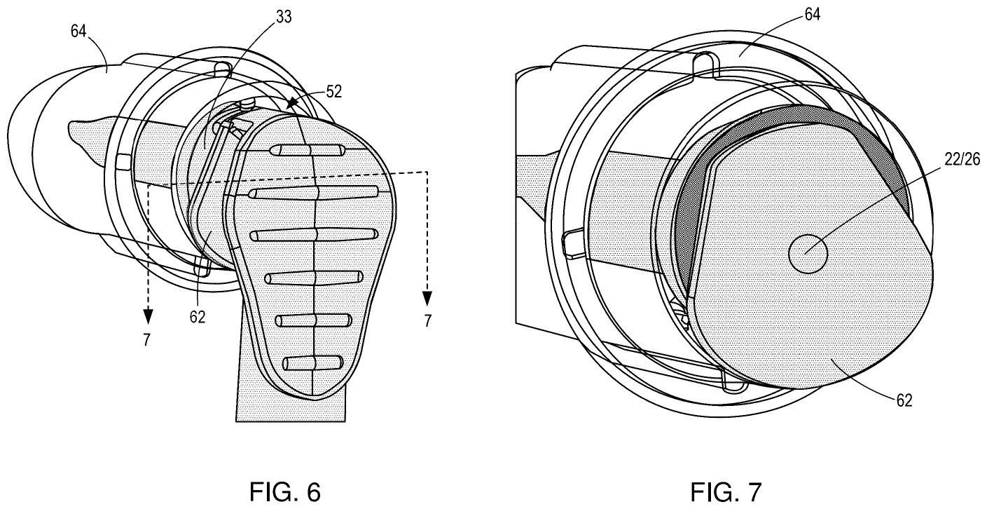

In one embodiment, and as best shown in FIGS. 4 and 5, the first segment 52 of joining member 20 includes a plurality of reinforcement ribs 58 formed thereon, with the reinforcement ribs 58 positioned at a connecting point of the connection spike 12 (i.e., end flange 33) and the first segment 52. The reinforcement ribs 58 may be spaced circumferentially about a central column 60 that joins a back end of the connection spike 12 with the first segment 52 of joining member 20. In some embodiments, the reinforcement ribs 58 comprise a first rib 58a and a second rib 58b on opposing sides of the central column 60, with each of the ribs 58 having a wedge-shaped construction, such as shown in FIG. 5. The reinforcement ribs 58 extend radially outward from the central column 60 to provide a connection of increased strength between the connection spike 12 and the joining member 20. The reinforcement ribs 58 function to increase the mechanical strength of the infusion adapter 10 and make the connection spike 12 more resistant to bending as the connection spike 12 is inserted/spiked to an IV bag through a rubber bung in an IV bag port.

In one embodiment, the first segment 52 of joining member 20 may also include a rest plate 62 formed thereon, at a location along a length of the first segment 52 between the reinforcement ribs 58 and the junction 56. In one embodiment, the rest plate 62 is formed adjacent the end flange 33 of connection spike 12 and is oriented parallel thereto, with the reinforcement ribs 58 of first segment 52 extending between the end flange 33 and the rest plate 62—such that the rest plate 62 is spaced apart from the end flange 33. In use of infusion adapter 10, the rest plate 62 may function as a datum for a spike cap 64 of infusion adapter 10 that covers the connection spike 12 prior to use of the infusion adapter 10, such as shown in FIG. 1 and in more detail in FIGS. 6 and 7. The datum provided by rest plate 62 ensures consistent assembly of the spike cap 64 with the infusion adapter 10 during manufacturing, so as to minimize the risk of the spike cap 64 becoming detached from the remainder of the infusion adapter 10 (i.e., detaching from connection spike 12). Additionally, with the rest plate 62 spaced apart from the end flange 33, a sufficient gap is provided that allows for gas sterilization (e.g., ethylene oxide (EO) sterilization) of the infusion adapter 10—including within an interior of the spike cap 64 after connection thereof to connection spike 12—with the gap enabling a flow of sterilization gas into the spike cap 64, to ensure proper sterilization of connection spike 12.

In one embodiment, the second segment 54 of joining member 20 includes an elongated portion 66 and a flanged divider 40. The elongated portion 66 generally extends a full length of the second segment 54, with one end thereof joined with the first segment 52 of joining member 20 and the other end thereof joined with flanged divider 40. The flanged divider 40 is affixed to the elongated portion 66 on one face/side thereof and to body 34 of connection stick 16 on the other face/side thereof, with the flanged divider 40 generally separating the joining member 20 from the connection stick 16. According to embodiments, the elongated portion 66 of second segment 54 is formed as a tapered member. The tapered elongated portion 66 flares radially outward along a length thereof as the elongated portion 66 extends from the end thereof joined with the first segment 52 to the end thereof joined with divider flange 40. The outwardly inclined angle of the elongated portion 66 provides for a thickening of an outer wall 68 thereof (that defines a portion of second fluid channel 26), to improve the strength and bending resistance of the joining portion 20, while also improving mold flow during molding of the infusion adapter 10.

As previously discussed, intravenous therapy applications allow patients to receive infusion and medication treatment. For example, therapy may include the administration of medications by IV using intravenous and subcutaneous or hypodermis routes, i.e., into the bloodstream and under the skin. Examples of medical treatments that intravenous therapy applications may provide to a patient include antibiotics, pain management medications, cancer treatments, and similar medications.

When performing infusion, it is often necessary to inject a drug or other medical substance into an infusion fluid located inside an infusion bag (i.e., container IV bag, as referred to hereafter) or other infusion fluid. This is often done by means of penetrating a septum or fluid barrier member (e.g., rubber bung) of an injection port on the IV bag.

Referring to FIG. 8, a fluid transfer system 100 may be provided that includes infusion adapter 10, a syringe assembly 102 (including a syringe 104 filled with medication fluid and a syringe adapter 45), intravenous (IV) tubing 106, and an IV bag 108 containing infusion fluid. The fluid transfer system 100 is configured to exchange fluids to/from the IV bag 108, such as by transferring medication fluid from the syringe assembly 102 through the infusion adapter 10 and into the infusion fluid of the IV bag 108, and then delivering the mixture of medication fluid and infusion fluid from the IV bag 108 to a patient via the IV tubing 106.

When a treatment is needed, a patient or a medical practitioner is able to spike or pierce a fluid barrier member 110 of a (first) injection port 112 on the IV bag 108 with the puncturing point 32 of connection spike 12 of infusion adapter 10.

With infusion adapter 10 securely connected to injection port 112 of infusion fluid container 102 via connection spike 12, a patient or a medical practitioner is able to connect the syringe assembly 102 to the infusion adapter 10. In exemplary embodiments, the syringe assembly 102 is connected to infusion adapter 10 via a pushing engagement of the syringe adapter 45 with the connection stick 16—with the connection stick 16 received within a housing 114 of the syringe adapter 45 (i.e., within a cavity 115 of the housing 114). Upon engagement of the syringe adapter 45 with the connection stick 16, the membrane 44 of the connection stick 16 engages a membrane 116 seated in the cavity 115 of housing 114 of syringe adapter 45, with the engaging of the membranes 44, 116 providing a substantially leak-free connection between the syringe adapter 45 and the infusion adapter 10 during transfer of the medication fluid from the syringe 104 to the IV bag 108.

With syringe assembly 102 connected to infusion adapter 10, the medication fluid contained in syringe 104 can be injected into the IV bag 108 via infusion adapter 10. In some embodiments, the syringe assembly 102 may then be disconnected from the infusion adapter 10.

With the medication fluid injected into the infusion fluid contained within IV bag 108, the fluid mixture is ready to be administered to a patient. For delivery of the fluid mixture to the patient, the IV tubing 106 may be connected to a second port 118 of infusion adapter 10 as shown in FIG. 13. The other end of the intravenous tubing 106 is fluidly connected to a bloodstream of the patient P, such as via a peripheral intravenous catheter (PIVC). In this manner, a medication may be administered to the patient intravenously.

Beneficially, embodiments of the invention thus are directed to an infusion adapter for use in a closed transfer of fluid, such as from a syringe containing a medication fluid therein to an intravenous fluid container. The infusion adapter includes a connection stick that provides for a simplified connection of a syringe assembly to the infusion adapter, such as via a simple push connection of the connection stick to the syringe assembly. The infusion adapter includes a plurality of structural features that increase the mechanical strength of the infusion adapter without increasing the size thereof and, in some cases, provide such improved mechanical strength while decreasing the volume of material needed to manufacture the adapter, such as via a molding manufacturing technique. The infusion adapter may thus resist bending or deformation during use thereof, such as when inserting/spiking the connection spike of the infusion adapter through the rubber bung of an IV bag connection port.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. An infusion adapter for connection with an infusion fluid container, the infusion adapter comprising:
a connection spike positioned at a first end of the infusion adapter, the connection spike having a main spike portion and a puncturing point configured to engage an injection port of an infusion fluid container;
a connection stick positioned at a second end of the infusion adapter and arranged orthogonal to the connection spike, the connection stick comprising a locking mechanism including a biasing member and an engagement member, with the locking mechanism configured to connect to an external component via a push-type engagement; and
a joining member connecting the connection spike to the connection stick in the orthogonal arrangement, the joining member integrally formed with the connection spike and the connection stick;
wherein a fluid path is formed through the joining member, the connection spike, and the connection stick, extending from the first end to the second end, to provide for a transfer of fluid injected from the second end to the first end.

2. The infusion adapter of claim 1, wherein the biasing member comprises a pair of cantilever arms and the engagement member comprises a projection extending radially outwards from each of the cantilever arms, the pair of cantilever arms configured to deflect radially inward during engagement of the connection stick to the external component.

3. The infusion adapter of claim 2, wherein the joining member is structured as an L-shaped member and comprises:
a first segment aligned with and joined to the connection spike; and
a second segment aligned with and joined to the connection stick;
wherein the first segment and second segment are connected at a junction.

4. The infusion adapter of claim 3, wherein the connection spike comprises an end flange on an end thereof opposite the connection spike, and wherein the first segment comprises a central column joined to the end flange, with the central column including a plurality of reinforcement ribs formed thereon that extend radially outward from the central column, the reinforcement ribs increasing a mechanical strength a connection between the joining member and the connection spike.

5. The infusion adapter of claim 4, wherein the plurality of reinforcement ribs comprises a first rib and a second rib positioned on opposing sides of the central column, with each of the first rib and the second rib having a wedge-shaped construction.

6. The infusion adapter of claim 4, wherein the first segment comprises a rest plate located along a length of the first segment between the plurality of reinforcement ribs and the junction, the rest plate oriented in parallel with the end flange and spaced apart therefrom by a gap provided by the central column and the plurality of reinforcement ribs.

7. The infusion adapter of claim 6, wherein the infusion adapter comprises a spike cap positioned over the connection spike, to protect the puncturing point, and wherein the rest plate provides a datum for the spike cap, to ensure proper connection of the spike cap onto the connection spike.

8. The infusion adapter of claim 7, wherein the gap is sized to allow for entry of a sterilizing gas into an interior of the spike cap with the spike cap positioned over the connection spike, so as to provide for sterilizing of the connection spike.

9. The infusion adapter of claim 3, wherein the second segment comprises an elongated portion and a flanged divider, the elongated portion generally extending a full length of the second segment, with one end thereof joined with the first segment and another end thereof joined with the flanged divider.

10. The infusion adapter of claim 9, wherein the flanged divider is affixed to the elongated portion on one face thereof and to the connection stick on an opposing face thereof.

11. The infusion adapter of claim 9, wherein the elongated portion comprises a tapered member that flares outward along a length thereof as the elongated portion extends from the end thereof joined with the first segment to the end thereof joined with the divider flange.

12. The infusion adapter of claim 11, wherein a thickness of an outer wall of the elongated portion increases along the length thereof as the elongated portion extends from the end thereof joined with the first segment to the end thereof joined with the divider flange.

13. The infusion adapter of claim 1, wherein the connection stick comprises a membrane seat that retains a membrane therein, the membrane providing for a closed transfer of fluid when the connection stick is connected to the external component.

14. The infusion adapter of claim 1, wherein the infusion adapter comprises an integrally formed, molded adapter.

15. A fluid transfer system comprising:
an infusion fluid container containing an infusion fluid therein, the infusion fluid container comprising a first port and a second port each having a fluid barrier member therein;
an intravenous tubing connected to the second port such that the intravenous tubing is in fluid communication with the infusion fluid container;
the infusion adapter of claim 1, with the connection spike engaged with the first port such that the infusion adapter is in fluid communication with the infusion fluid container; and
a syringe assembly coupled to the connection stick, the syringe assembly comprising a pre-filled syringe containing medication fluid and a syringe adapter, with the connection stick engaged with the syringe adapter.

16. The fluid transfer system of claim 15, wherein the fluid barrier member comprises a rubber bung.

17. The fluid transfer system of claim 15, wherein the syringe adapter comprises a housing defining a cavity within which the locking mechanism is received upon connection of the connection stick with the syringe adapter.

18. The fluid transfer system of claim 17, wherein the syringe adapter comprises another membrane, the another membrane seated in the cavity of the housing and configured to engage the membrane of the connection stick upon connection of the syringe assembly to the infusion adapter, with the engaging of the membrane and the another membrane providing a substantially leak-free connection of the infusion adapter with the syringe adapter during transfer of the medication fluid.
